(19) United States
(12) Patent Application Publication
(10) Pub. No.: US 2007/0082039 A1
(43) Pub. Date: Apr. 12, 2007

(54) Synthesis of fatty alcohol esters of alpha-hydroxy carboxylic acids, the use of the same as percutaneous permeation enhancers, and topical gels for the transdermal delivery of steroids

(76) Inventors: Gerald S. Jones JR., Norwood, MA (US); Joseph P. St. Laurent, Lakeville, MA (US); Scott A. Goodrich, Stoughton, MA (US)

Correspondence Address: Mark J. Pandiscio;Pandiscio & Pandiscio, P.C., 470 Totten Pond Road, Waltham, MA, 02451-1914, US

(21) Appl. No.: 11/527,957

(22) Filed: Sep. 27, 2006

Related U.S. Application Data
(63) Continuation-in-part of application No. 11/251,738, filed on Oct. 17, 2005.
(60) Provisional application No. 60/619,887, filed on Oct. 18, 2004.
(60) Provisional application No. 60/698,248, filed on Jul. 11, 2005.
(60) Provisional application No. 60/720,792, filed on Sep. 27, 2005.

(51) Int. Cl. A61K 31/56; A61K 9/70

(52) U.S. Cl. 424/449; 514/171

(57) ABSTRACT

The present invention provides a novel approach for the preparation of fatty alcohol esters of α-hydroxy carboxylic acids. The present invention also provides a novel topical gel for the transdermal delivery of steroids using the fatty alcohol esters as permeation enhancers.

### REFERENCE TO PENDING PRIOR PATENT APPLICATIONS

This patent application:

(1) is a continuation-in-part of pending prior U.S. patent application Ser. No. 11/251,738, filed Oct. 17, 2005 by Gerald S. Jones, Jr. et al. for SYNTHESIS OF FATTY ALCOHOL ESTERS OF α-HYDROXY CARBOXYLIC ACIDS AND THEIR USE AS PERCUTANEOUS ABSORPTION ENHANCERS (Attorney's Docket No. CHEMIC-4), which patent application in turn claims benefit of: (i) prior U.S. Provisional Patent Application Ser. No. 60/619,887, filed Oct. 18, 2004 by Gerald S. Jones, Jr. et al. for SYNTHESIS OF DODECYL LACTATE AND RELATED COMPOUNDS AND THEIR USE AS PERCUTANEOUS ABSORPTION ENHANCERS (Attorney's Docket No. CHEMIC-3 PROV); and (ii) prior U.S. Provisional Patent Application Ser. No. 60/698,248, filed Jul. 11, 2005 by Gerald S. Jones, Jr. et al. for SYNTHESIS OF DODECYL LACTATE AND RELATED COMPOUNDS AND THEIR USE AS PERCUTANEOUS ABSORPTION ENHANCERS (Attorney's Docket No. CHEMIC-4 PROV); and

(2) claims benefit of pending prior U.S. Provisional Patent Application Ser. No. 60/720,792, filed Sep. 27, 2005 by Gerald S. Jones, Jr. et al. for NOVEL TOPICAL GEL PRODUCT FOR THE TRANSDERMAL DELIVERY OF STEROIDS (Attorney's Docket No. CHEMIC-5 PROV).

The four above-identified patent applications are hereby incorporated herein by reference.

### FIELD OF THE INVENTION

This invention relates to transdermal drug delivery in general, and more particularly to the synthesis of fatty alcohol esters of α-hydroxy carboxylic acids, their use as percutaneous permeation enhancers, and to topical gels for the transdermal delivery of steroids.

### BACKGROUND OF THE INVENTION

Transdermal drug delivery (TDD) is the delivery of drugs by absorption through the skin and into the body. TDD has become an established, non-invasive approach for both the local and systemic administration of drugs.

TDD offers the advantages of smaller drug doses, improved efficacy, reduced toxicity, elimination of first-pass metabolism, minimization of pain, and possible sustained release, among others.

Despite the obvious advantages of TDD, this delivery approach has not been more widely exploited due to the intrinsic barrier properties of the skin. More particularly, human skin is made up of two layers, the epidermis (i.e., the outer layer) and the dermis (i.e., the inner layer). The outermost layer of the epidermis is the stratum corneum. The stratum corneum is the main barrier to drug delivery. If an improved means were available for penetrating the stratum corneum barrier, TDD would become a more attractive drug delivery option.

It has been discovered that the integrity of the stratum corneum can be disrupted (and hence its permeability increased) through the use of sound energy, electrical energy or physical methods, including chemical treatment. Considerable effort has been concentrated on identifying non-toxic chemical compounds that interact with the stratum corneum so as to increase the potential for drug penetration. These compounds are sometimes referred to as “permeation enhancers”, “penetration enhancers” or “absorption enhancers”.

A review of the recent patent literature reveals numerous practical examples of permeation enhancers used as transdermal delivery devices, including:

| U.S. Pat. No. | Year | Assignee | Title | Enhancer |
|---|---|---|---|---|
| 6699497 | 2004 | Alza | Formulations for the transdermal | lauryl lactate; |
| | | | administration of fenoldopam | myristyl lactate |
| 6638981 | 2003 | EpiCept | Topical compositions and | Transcutol ® P |
| | | | methods for treating pain | |
| 6582724 | 2003 | Derma- | Dual enhancer composition for | Transcutol ® P; |
| | | trends | topical and transdermal drug | Azone ® |
| | | | delivery | |
| 6156753 | 2000 | Vivus | Local administration of type III | Azone ®; |
| | | | phosphodiesterase inhibitors for | SEPA ® |
| | | | the treatment of erectile | |
| | | | dysfunction | |
| 6118020 | 2000 | NexMed | Crystalline salts of dodecyl 2- | NexACT ® |
| | | | (N,N-dimethylamino)propionate | |
| 6004578 | 1999 | Alza | Permeation enhancers for | lauryl acetate; |
| | | | transdermal drug delivery | lauryl lactate |
| | | | compositions, devices and | |
| | | | methods | |
| 5843468 | 1998 | Alza | Skin permeation enhancer | glycerol |
| | | | compositions comprising glycerol | monolaurate; |
| | | | monolaurate and lauryl acetate | lauryl acetate |
| 5314694 | 1994 | Alza | Transdermal formulations, methods and | lauryl lactate |
| | | | devices | |

Approximately thirty chemical compounds have been routinely used by the pharmaceutical industry as permeation enhancers. Most of these compounds, however, have been found to provide only a slight improvement in drug absorption.

The known and putative permeation enhancers include members of several classes of organic compounds, including:

| Class | Compound(s) |
|---|---|
| alcohols | ethanol; |
| | isopropanol; |
| | benzyl alcohol |
| glycols | propylene glycol; |
| | diethylene glycol monoethyl ether (Transcutol ®) |
| glycol esters | glycerol monolaurate |
| fatty acids | oleic acid |
| fatty acid esters | isopropyl myristate |
| fatty alcohol esters | lauryl lactate; |
| | myristyl lactate; |
| | cetyl lactate; |
| | dodecyl methacrylate |
| miscellaneous | DMSO; |
| | laurocapram (Azone ®); |
| | 2-nonyl-1,3-dioxolane (SEPA ®); |
| | dodecyl 2-(N,N-dimethylamino) propionate |
| | (NexACT ®) |

Most of the known permeation enhancers have a common general structure, which is that of a non-ionic surface-active agent, i.e., a non-ionic surfactant. This general structure consists of two discrete portions that possess diametrically-opposed physicochemical properties: a polar head and a lipophilic tail. The polar head of the molecule is hydrophilic (i.e., water absorbable) and can include one of a variety of functional groups, such as those listed above (e.g., alcohols, glycols, glycol esters, etc.). The lipophilic tail is hydrophobic (i.e., non-water absorbable) and consists of a hydrocarbon chain that typically ranges from eight to sixteen carbon atoms in length. FIG. 1 illustrates the structures of eight permeation enhancers (both proprietary and generic) having the aforementioned polar head and lipophilic tail structure.

Of the chemical compounds used as permeation enhancers, one class of compounds in particular (i.e., fatty alcohol esters of α-hydroxy carboxylic acids) has found widespread use in cosmetic and pharmaceutical formulations as humectants and/or emollients. Among these fatty alcohol esters of α-hydroxy carboxylic acids, alkyl lactates are the most widely used, particularly those where the alkyl group is greater than eight carbon atoms in length (i.e., >C₈). Of these alkyl lactates having a carbon backbone greater than 8, lauryl lactate (also known as dodecyl lactate), myristyl lactate and cetyl lactate have generated considerable interest for TDD applications due to their permeation-enhancing properties.

Esters (including the aforementioned fatty alcohol esters) can be prepared using a variety of conventional processes. These conventional processes typically utilize a carboxylic acid (e.g., α-hydroxy carboxylic acid) and alcohol to produce the desired ester. The following briefly discusses conventional processes for preparing esters.

### Preparation Of Esters

Looking next at FIG. 2, most industrial processes for the preparation of esters (i.e., esterification) involve the reaction of a carboxylic acid 5 with an alcohol 10 in the presence of a chemical esterification catalyst 15 to produce an ester 20. Chemical esterification catalyst 15 is generally an acid or a base, e.g., an organic sulfonic acid or a metal alkylate. One problem with the ester-producing mechanism shown in FIG. 2 is that the esters produced by this process typically contain chemical catalyst residues and various by-products, including difficult-to-remove ethers.

Alternatively, the esterification of a carboxylic acid with an alcohol can be accomplished via an enzymatic process. In this reaction, which is similar to the one illustrated in FIG. 2, an enzyme (most often a lipase) is used in place of the chemical esterification catalyst 15, typically resulting in a cleaner reaction and, possibly, a product of somewhat higher purity. European Patent No. 0383405 describes the use of a lipase as an enzymatic catalyst in the esterification of C₇-C₃₆ monocarboxylic or dicarboxylic acids with C₂-C₈ alcohols.

FIG. 3 shows a Fischer-type esterification process, where carboxylic acid is converted into an ester in the presence of alcohol and an acidic chemical catalyst. In FIG. 3, the Fischer-type esterification process is illustrated in the context of producing the fatty alcohol ester lauryl lactate. More particularly, an α-hydroxy carboxylic acid 25 (e.g., lactic acid, where R═CH₃; α-hydroxypropionic acid) is combined with an alcohol 30 (e.g., dodecanol, where R′=C₁₂H₂₅) in the presence of a chemical esterification catalyst 33 so as to produce lauryl lactate 35 (where R is CH₃ and R′ is C₁₂H₂₅). This is believed to be a common commercial approach for producing lauryl lactate.

One problem with the reaction shown in FIG. 3 is the potential reactivity of the α-hydroxy group of the α-hydroxy carboxylic acid 25. As a competitive nucleophile in the esterification reaction, involvement of the α-hydroxy group can result in the formation of a polyester, e.g., the polyester 40 shown in FIG. 3. Subsequently, lactonization of the polyester 40 may in turn produce a cyclic ester, e.g., the cyclic ester 45 shown in FIG. 3. Production of polyester 40 and cyclic ester 45 can reduce the purity of the target lauryl lactate 35.

Another problem with the Fischer-type esterification process shown in FIG. 3 is that the lauryl lactate 35 produced by this process can contain as much as 5% dodecanol, as well as intermolecular esterification products (typically originating from the commercial lactic acid 25) and varying amounts of unidentified polymeric species intermixed in the product.

While the lauryl lactate produced by the Fischer-type esterification process of FIG. 3 is relatively inexpensive to produce, the limited purity of the lauryl lactate is directly related to the use of commercial lactic acid as a starting material in the esterification process. Commercial lactic acid is typically available in an aqueous solution (˜85% lactic acid); however, this commercial lactic acid also commonly contains varying amounts of intermolecular esterification products. When subjected to the relatively harsh reaction conditions of chemical catalyst esterification, a complex reaction mixture is inevitable, and often results in the formation of various undesirable by-products.

While esterification of lactic acid with dodecanol can also be accomplished via an arguably milder enzymatic process (as opposed to the chemical catalyst process of FIG. 3), this enzymatic process still necessitates the use of commercially-available lactic acid as a starting material. Therefore, this process still produces a low-purity lauryl lactate product.

A review of the literature reveals several other approaches for producing alkyl lactates (including lauryl lactate).

For example, the synthesis of lauryl lactate using a Dawson phosphotungstic acid catalyst is described in Guangzhou Huaxue, 2002, 27(1), 32-33, 55. The abstract of this article reported that, under optimum conditions, the yield of lauryl lactate was greater than 93%. However, the purity of the product was not reported.

In another example, described in Xiamen Daxue Xuebao, Ziran Kexueban, 1997, 36(4), 581-584, various lactate esters were prepared by a Fischer-type esterification process. Yields ranged from 81-98%, but the yield for lauryl lactate was not specifically reported in the abstract. Furthermore, the purity of the various lactate esters was not reported.

And, in another example, esters of α-hydroxy carboxylic acids were prepared by an enzymatic process, using a lipase as the enzyme. This study was described in Enzyme and Microbial Technology, 1999, 25, 745-752, and it focused on the optimization of reaction conditions for the syntheses of lactate and glycolate esters of fatty alcohols (including lauryl lactate) via esterification of carboxylic acid. Optimization of the process resulted in a high yield of lauryl lactate (˜96%) from lactic acid and dodecanol. However, the purity of the lauryl lactate product was not reported. Spanish Patent No. ES 2143940 is believed to relate to this study.

While all of the foregoing approaches are capable of producing esters, including fatty alcohol esters and including specifically lauryl lactate, they all produce a relatively impure product. This relative impurity has proven to be a problem for many applications, including the use of alkyl lactates (e.g., lauryl lactate, cetyl lactate and myristyl lactate) as permeation enhancers for TDD.

By way of example, commercially-available lauryl lactate is relatively easily available, inexpensive, and, according to the manufacturers' claims, of reasonable purity. For example, one currently available lauryl lactate product is typically reported to be 95% lauryl lactate, with the major impurities being dodecanol (4%) and lactic acid (1%). However, chromatographic data generated by several independent laboratories clearly show that the commercial products are much less pure than reported and, in many cases, are <70%.

Thus, the current approaches for the synthesis of alkyl lactates yield products of low purity. This in turn significantly limits the effectiveness of these compounds as permeation enhancers.

As a result, there is a need for a convenient, efficient and scalable process for the synthesis of high-purity alkyl lacates, whereby to produce permeation enhancers of increased effectiveness.

### Use Of TDD In The Treatment Of Hypogonadism And Erectile Dysfunction

Untreated hypogonadism in men can have serious medical consequences, including: (i) reduced bone density; (ii) reduced muscle mass; (iii) increased cardiovascular risk (secondary to increased abdominal fat); (iv) undesirable effects on mood, behavior, and cognitive function; and (v) undesirable effects on sexual function, including erectile dysfunction (ED). Male erectile dysfunction is defined as the inability to achieve or maintain an erection sufficient for sexual performance. According to the 1992 NIH Consensus Statement, an estimated 10-20 million males in the United States alone, typically 40-70 years of age, suffer from ED. There is a strong association between ED and age and, in view of the aging U.S. population, the prevalence of ED in the United States is likely to increase.

A current treatment for hypogonadism is testosterone replacement therapy. The Endocrine Society estimates that only 5% of hypogonadal men in the United States are currently receiving treatment for their condition.

Various testosterone replacement treatments are currently available. These include: (i) intramuscular injections; (ii) oral replacement; (iii) pellet implants; and (iv) transdermal patches. About four years ago, a 1% testosterone gel (AndroGel®, by Solvay) was released to the market. This testosterone gel is free of many of the disadvantages associated with the other testosterone replacement methods, and its rapid acceptance has resulted in annual sales in the United States alone of an estimated $500 million.

### AndroGel®

AndroGel® is a hydroalcoholic gel containing 1% testosterone as the active pharmacological ingredient. The pharmacologically inactive ingredients in AndroGel® are ethanol (67%), purified water, sodium hydroxide, carbomer (Carbopol® 980) and isopropyl myristate. Isopropyl myristate acts as a permeation enhancer for the AndroGel® product. AndroGel®, is believed to comprise the following substances in the approximate following amounts:

| Substance | % w/w |
|---|---|
| Ethanol | 67.0 |
| Water | 31.58 |
| Isopropyl myristate | 0.50 |
| Testosterone | 1.00 |
| Carbopol ® 980 | 0.90 |
| NaOH | ˜0.02 |
| | 100.00 |

As noted above, isopropyl myristate is included in the AndroGel® product as a permeation enhancer. Isopropyl myristate is a lower alcohol ester of a fatty acid. Specifically, it is the ester made from isopropanol (a C3 alcohol) and tetradecanoic acid (a C14 fatty acid, also known as myristic acid). By comparison, lauryl lactate (which, as noted above, is also a permeation enhancer) is a fatty alcohol ester of a short-chain carboxylic acid. Each compound is a fatty ester, i.e., a portion of each compound consists of a highly lipophilic (“fatty”) saturated hydrocarbon chain. The structural differences between the two esters (i.e., isopropyl myristate and lauryl lactate) are depicted in FIG. 4.

### Other Permeation Enhancers And Testosterone Replacement Products

A second generation of permeation enhancers, which are reportedly more efficacious than those produced in the past, are currently under development by several companies. Notable among this second generation of permeation enhancers are Azone®, NexAct®, and SEPA®. SEPA® (Soft Enhancement of Percutaneous Absorption) is actually an entire family of permeation enhancers developed by MacroChem Corporation.

MacroChem has used its SEPA® permeation enhancers to produce a more effective testosterone gel. More particularly, MacroChem's first-generation hypogonadism treatment was a hydroalcoholic gel formulated with 1% testosterone and 5% SEPA® 0009 (2-n-nonyl-1,3-dioxolane). In a three-way, randomized, double-blind human pharmacokinetic study involving 15 men at three sites, it was found that 2.5 grams of the SEPA®-containing product provided an effectiveness comparable to 5 grams of AndroGel®. However, since testosterone absorption from the SEPA®-containing product was rapid and demonstrated levels above baseline for 10-12 hours post-application, the formulation was abandoned in favor of a cream, Opterone®.

### SUMMARY OF THE INVENTION

One object of the present invention is to provide a new and improved method for making fatty alcohol esters of α-hydroxy carboxylic acids.

Another object of the present invention is to provide a new and improved method for making fatty alcohol esters of α-hydroxy carboxylic acids, wherein the ester is an alkyl lactate.

Still another object of the present invention is to provide a new and improved method for making fatty alcohol esters of α-hydroxy carboxylic acids, wherein the ester is lauryl lactate, cetyl lactate and/or myristyl lactate.

Yet another object of the present invention is to provide a method for making fatty alcohol esters of α-hydroxy carboxylic acids that is convenient, efficient, reproducible, and scalable.

And another object of the present invention is to provide a method for making fatty alcohol esters of α-hydroxy carboxylic acids, wherein the purity of the product is consistently greater than 95%.

And still another object of the present invention is to create a novel family of compounds for use as permeation enhancers.

And yet another object of the present invention is to create a novel product utilizing such permeation enhancer compounds for the improved transdermal delivery of steroids.

These and other objects are addressed by the provision and use of the present invention, which provides a novel approach for the preparation of fatty alcohol esters of α-hydroxy carboxylic acids, as well as their use as a permeation enhancers for the improved transdermal delivery of steroids.

In one form of the invention, the target fatty alcohol ester of α-hydroxy carboxylic acid is produced by converting a lower alkyl ester of α-hydroxy carboxylic acid into a fatty alcohol ester of α-hydroxy carboxylic acid via alcoholysis (i.e., transesterification). The transesterification process is an equilibrium reaction, catalyzed chemically (i.e., with acids or bases) or enzymatically (e.g., with lipases, etc.), that is shifted in the desired direction so as to produce the desired product. One preferred way of shifting the reaction in the direction of the desired product is by reducing the concentration of one of the products (e.g., by distillation of a lower-boiling alcohol as soon as it is formed). Another preferred way of shifting the reaction in the direction of the desired product is by increasing the concentration of one of the reactants (e.g., by adding more of the starting ester).

In one form of the invention, there is provided a gel comprising:

a steroid; and

a permeation enhancer to facilitate the transdermal delivery of the steroid, wherein the permeation enhancer comprises a high-purity fatty alcohol ester.

In another form of the invention, there is provided a topical transdermal drug delivery product comprising:

a drug; and

a permeation enhancer to facilitate the transdermal delivery of the drug, wherein the permeation enhancer comprises a high-purity fatty alcohol ester.

In another form of the invention, there is provided a method for manufacturing a transdermal steroid delivery product, comprising:

(i) providing a steroid and providing a permeation enhancer to facilitate the transdermal delivery of the steroid, wherein the permeation enhancer comprises a high-purity fatty alcohol ester; and

(ii) combining the steroid and the fatty alcohol ester in a gel.

In another form of the invention, there is provided a method for manufacturing a transdermal drug delivery product, comprising:

(i) providing a drug and providing a permeation enhancer to facilitate the transdermal delivery of the drug, wherein the permeation enhancer comprises a high-purity fatty alcohol ester; and

(ii) combining the drug and the fatty alcohol ester in a mass selected from the group consisting of a gel, a cream, an ointment, a liquid, a colloid, a semi-solid, and a solid.

In another form of the invention, there is provided a method for delivering a steroid to living tissue, comprising:

(i) providing a gel comprising (a) a steroid, and (b) a permeation enhancer to facilitate the transdermal delivery of the steroid, wherein the permeation enhancer comprises a high-purity fatty alcohol ester; and

(ii) applying the gel to the living tissue.

In another form of the invention, there is provided a method for delivering a drug to living tissue, comprising:

(i) providing a mass comprising (a) a drug, and (b) a permeation enhancer to facilitate the transdermal delivery of the drug, wherein the permeation enhancer comprises a high-purity fatty alcohol ester, and further wherein the mass is selected from the group consisting of a gel, a cream, an ointment, a liquid, a colloid, a semi-solid, and a solid; and

(ii) applying the mass to the living tissue.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 shows the structures of eight known permeation enhancers;

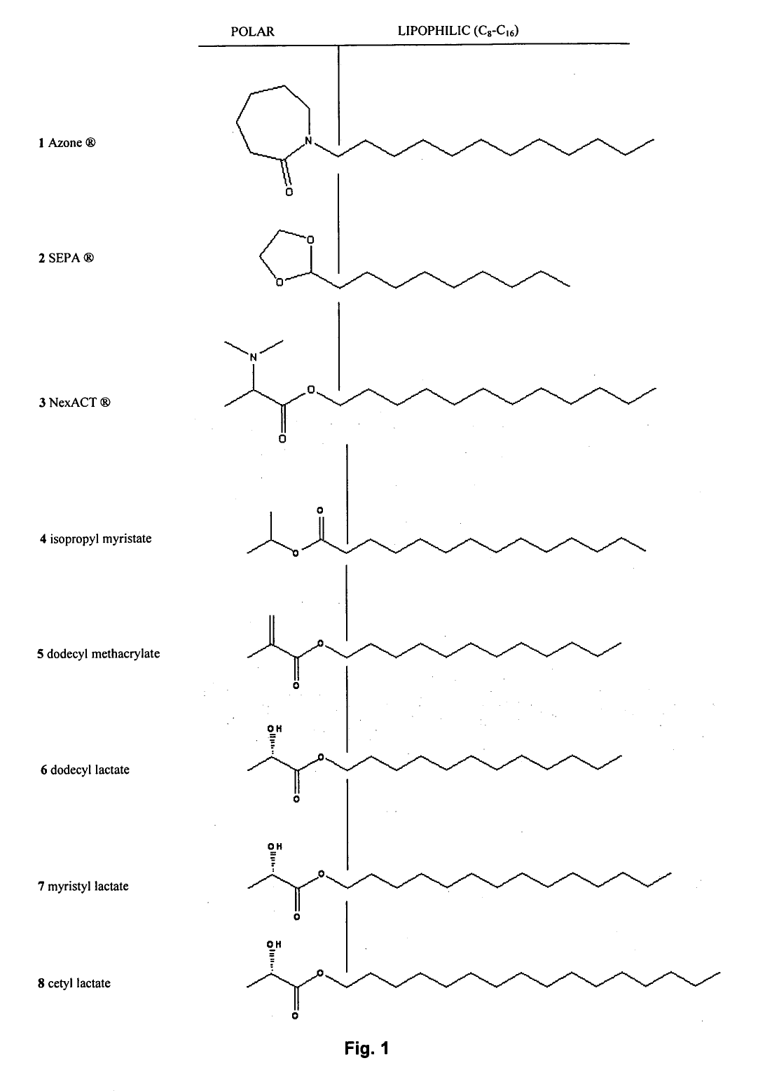

FIG. 2 is an illustration of a process for the preparation of esters using a chemical esterification catalyst (e.g., an organic sulfonic acid or a metal alkylate);

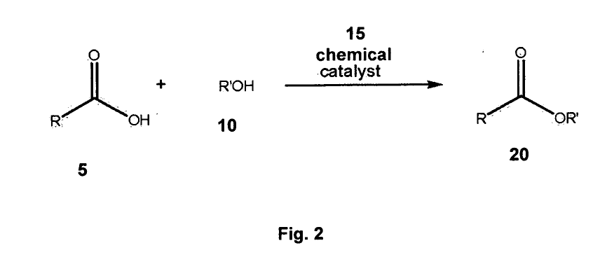

FIG. 3 shows a Fischer-type esterification process where an α-hydroxy carboxylic acid is converted into an ester in the presence of alcohol and a chemical esterification catalyst;

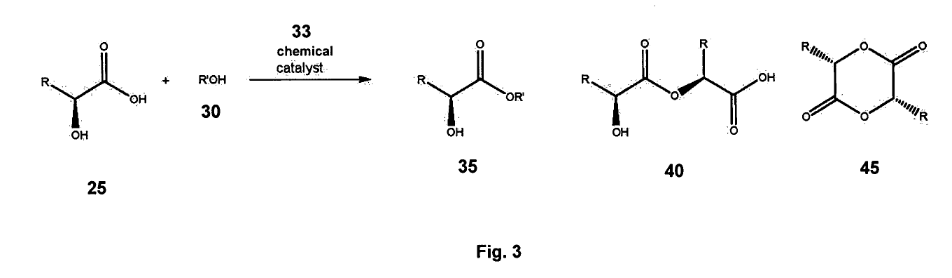

FIG. 4 shows a structural comparison of isopropyl myristate and lauryl lactate;

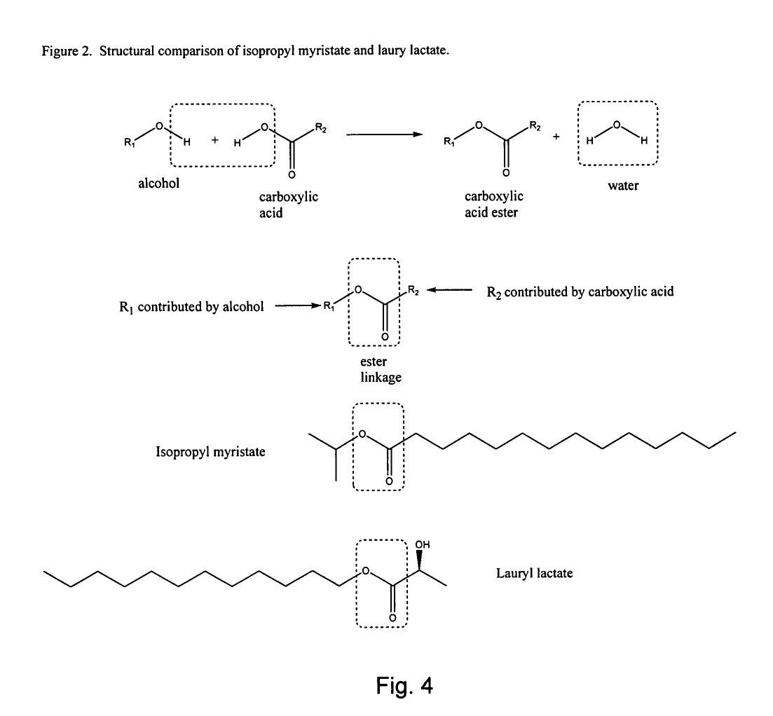

FIG. 5 illustrates a transesterification process in general;

FIG. 6 shows the transesterification process of the present invention; and

FIG. 7 shows the increased permeation enhancement achieved by a testosterone gel formed in accordance with the present invention.

### DETAILED DESCRIPTION OF THE INVENTION

The present invention provides a novel approach for the preparation of fatty alcohol esters of α-hydroxy carboxylic acids, their use as permeation enhancers for the improved transdermal delivery of steroids, and novel topical gels for the transdermal delivery of steroids.

### Preparation Of Fatty Alcohol Esters Of α-Hydroxy Carboxylic Acids

In one form of the present invention, the target fatty alcohol ester of α-hydroxy carboxylic acid is produced by converting one ester into another ester via alcoholysis (i.e., transesterification). The transesterification process is an equilibrium reaction, catalyzed chemically (i.e., with acids or bases) or enzymatically, that is shifted in the desired direction so as to produce the desired product. One preferred way of shifting the reaction in the direction of the desired product is by reducing the concentration of one of the products (e.g., by distillation of a lower-boiling alcohol as soon as it is formed). Another preferred way of shifting the reaction in the direction of the desired product is by increasing the concentration of one of the reactants (e.g., by adding more of the starting ester).

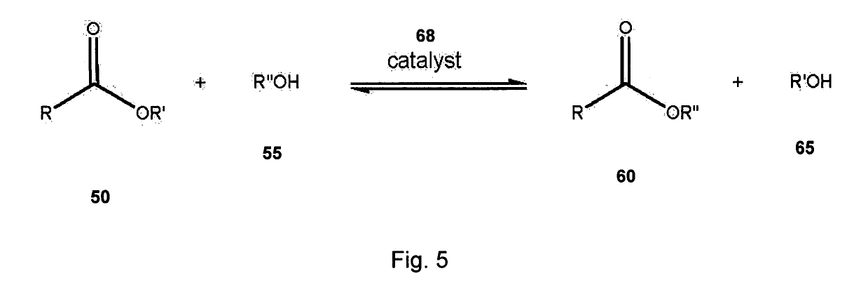

Looking next at FIG. 5, there is shown a general transesterification process. More particularly, in such a process, an ester 50 and an alcohol 55 are converted into an ester 60 and an alcohol 65 using a catalyst 68 to drive the reaction.

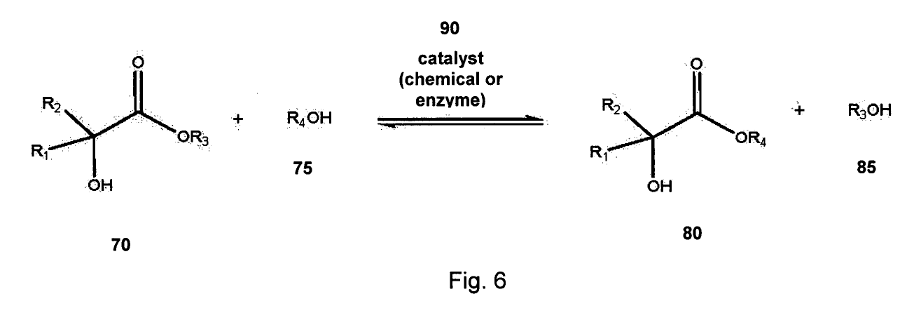

Looking next at FIG. 6, the present invention utilizes a novel transesterification process to produce the target fatty alcohol ester of α-hydroxy carboxylic acid. More particularly, lower-alkyl esters of α-hydroxy carboxylic acids 70 and primary or secondary alcohols 75 are converted into fatty alcohol esters 80 and an alcohol 85 using an appropriate chemical (i.e., acid or base) or enzyme catalyst 90 to catalyze the reaction.

The novel transesterification process of the present invention offers several distinct advantages:

(i) the starting reagents (i.e., lower-alkyl esters of α-hydroxy carboxylic acids 70 and alcohols 75) are relatively inexpensive, readily available, and of good quality and high purity;

(ii) the product fatty alcohol esters 80 are of high purity (i.e., >95%); and

(iii) the process is amenable to scaling (i.e., capable of volume production).

Various lower-alkyl esters of α-hydroxy carboxylic acids 70 may be used in this new process. Some preferred lower-alkyl esters of α-hydroxy carboxylic acids 70 are those where:

(i) R₁ is one of the following: H, straight chained or branched alkyl, cycloalkyl, substituted alkyl, arylalkyl, aryl, substituted aryl, and heteroaryl;

(ii) R₂ is either H or alkyl; and

(iii) R₃ is one of the following: C₁-C₄ (e.g., methyl, ethyl, 2,2,2-trifluoroethyl, vinyl, propyl, isopropyl, isopropenyl, butyl, isobutyl, sec-butyl or tert-butyl).

In particular, the ethyl esters of glycolic acid, lactic acid, mandelic acid, 2-hydroxyisobutyric acid, and 2-hydroxycaproic acid are preferred for the lower-alkyl ester of α-hydroxy carboxylic acid 70 used in the new process.

Various alcohols 75 may be used. Some preferred alcohols 75 are straight-chain alcohols where R₄ is an alkyl group greater than or equal to an eight carbon chain (i.e., ≧C₈). Other suitable alcohols can be primary or secondary, can be branched, can contain various substituents (other than hydroxyl), and can be monounsaturated or polyunsaturated.

In particular, fatty alcohols such as 1-dodecanol, 2-dodecanol, 1-tetradecanol, and 1-hexadecanol are preferred for the alcohols 75 in the new process.

Various chemicals (i.e., acids or bases) and enzymes may be used as the catalyst 90 in the new process. Some preferred enzymes are lipases. In particular, lipases obtained from the following well-known microorganisms are preferred: Aspergillus species, Rhizopus species, Penicillum species, Candida species, Pseudomonas species, Mucor species, and Humicola species.

It is preferred (but not necessarily required) that the lipase be immobilized by attachment to a suitable water-insoluble inorganic or organic material such as silica, ion exchange resins, acrylate resins, porous polystyrene, etc. Such immobilization of the lipase (i.e., by attachment to a suitable water-insoluble inorganic or organic material) is well known to those skilled in the art.

The transesterification process of the present invention is an equilibrium reaction, catalyzed chemically (i.e., with acids or bases) or enzymatically, that is shifted in the desired direction to produce the desired product. One preferred way of shifting the reaction in the direction of the desired product is by reducing the concentration of one of the products (e.g., by distillation of a lower-boiling alcohol as soon as it is formed). Another preferred way of shifting the reaction in the direction of the desired product is by increasing the concentration of one of the reactants (e.g., by adding more of the starting ester).

In one preferred form of the invention, the transesterification process is conducted in such a way that the alcohol 85 formed in the course of the reaction is removed from the reaction medium. Alcohol removal can be accomplished in a variety of ways apparent to those skilled in the art including, but not limited to, evaporation under ambient conditions, evaporation facilitated by heat, convection, inert gas flow, application of vacuum, distillation (including azeotropic and vacuum distillation), chemical or enzymatic modification, adsorption, etc.

The transesterification process of the present invention can also be accomplished by any technique that facilitates the interaction of the reactants and results in the formation of the target product (i.e., fatty alcohol esters 80) and the generation of alcohol 85.

In one embodiment of the present invention, the reactants (i.e., the lower-alkyl ester of α-hydroxy carboxylic acid 70 and the alcohol 75) are combined such that the amount of ester present relative to the amount of alcohol present is at least an equimolar amount, and may represent a two-to-tenfold or higher molar excess. The reactants are combined in a vessel of appropriate size and design such that controlled heating and stirring is allowed, and from which the reaction mixture can be easily removed. The catalyst (preferably an enzyme) 90 is added in an amount that is typically between about 0.10 to about 2 times the weight of the alcohol 75. The reactants are stirred at an appropriate speed for twelve to forty-eight hours with controlled heating from about 30-90° C.

In another embodiment of the present invention, the reactants (i.e., the lower-alkyl ester of α-hydroxy carboxylic acid 70 and the alcohol 75) are combined such that the amount of ester present relative to the amount of alcohol present is at least an equimolar amount, and may represent a two-to-tenfold or higher molar excess. The reactants are combined in a vessel of appropriate size and design such that controlled heating and stirring is allowed, and from which the reaction mixture can be easily removed. The catalyst (preferably an enzyme) 90 is added in an amount that is between about 0.10 to about 2.0 times the weight of the alcohol 75. In this embodiment of the present invention, a fourth substance, i.e., an absorbing agent (e.g., molecular sieves, silica gel, etc.), is added in an amount that is between two to five times the weight of the alcohol 75. The reactants are stirred at an appropriate speed for twelve to forty-eight hours with controlled heating from about 30-90° C.

In another embodiment of the present invention, the reactants (i.e., the lower-alkyl ester of α-hydroxy carboxylic acid 70 and the alcohol 75) are combined such that the amount of ester present relative to the amount of alcohol present is at least an equimolar amount, and may represent a two-to-tenfold or higher molar excess. The reactants are combined in a vessel of appropriate size and design such that controlled heating and stirring is allowed, and from which the reaction mixture can be easily removed. The catalyst (preferably an enzyme) 90 is added in an amount that is between about 0.10 to about 2.0 times the weight of the alcohol 75. An absorbing agent (e.g., molecular sieves, silica gel, etc.) may be added in an amount that is between two to five times the weight of the alcohol 75. The reactants are stirred at an appropriate speed for twelve to forty-eight hours with controlled heating from about 30-90° C. The alcohol 85 generated during the reaction is removed by distillation by reducing the pressure in the system through the application of a weak vacuum.

In another embodiment of the present invention, the reactants (i.e., the lower-alkyl ester of α-hydroxy carboxylic acid 70 and the alcohol 75) are combined such that the amount of ester present relative to the amount of alcohol present is at least an equimolar amount, and may represent a two-to-tenfold or higher molar excess. The reactants are combined in a vessel of appropriate size and design such that controlled heating and stirring is allowed, and from which the reaction mixture can be easily removed. In this embodiment of the present invention, a solvent is added in a volume of 100-1000 ml/mole of alcohol. Suitable solvents include, but are not limited to, acetone, acetonitrile, dioxane, heptane, hexanes, and tetrahydrofuran. The catalyst (preferably an enzyme) 90 is added in an amount that is between about 0.10 to about 2.0 times the weight of the alcohol 75. An absorbing agent (e.g., molecular sieves, silica gel, etc.) may be added in an amount that is between two to five times the weight of the alcohol 75. The reactants are stirred at an appropriate speed for twelve to forty-eight hours with controlled heating from about 30-90° C. The alcohol 85 generated during the reaction is removed by co-distillation with the aforementioned solvent by reducing the pressure in the system through the application of a weak vacuum. Additional amounts of the aforementioned solvent are added to the system at approximately the same rate as distillate is formed.

In another embodiment of the present invention, the reactants (i.e., the lower-alkyl ester of α-hydroxy carboxylic acid 70 and the alcohol 75) are combined in a vessel of appropriate size and design such that controlled heating and stirring is allowed, and from which the reaction mixture can be easily removed. The alcohol 75 and catalyst (preferably an enzyme) 90 are combined such that the catalyst 90 is present in an amount that is between about 0.10 to about 2.0 times the weight of the alcohol 75. A solvent is added in a volume of 100-1000 ml/mole of alcohol. Suitable solvents include, but are not limited to, acetone, acetonitrile, dioxane, heptane, hexanes, and tetrahydrofuran. An absorbing agent (e.g., molecular sieves, silica gel, etc.) may be added in an amount that is between two to five times the weight of the alcohol 75. The reactants are stirred at an appropriate speed with controlled heating from about 30-90° C. while reducing the pressure in the system through the application of a weak vacuum. The ester 70 is added slowly by means of an addition funnel or syringe pump until an equimolar amount or slight molar excess of ester has been added. The alcohol 85 generated during the reaction is removed by co-distillation with the aforementioned solvent. Additional amounts of the aforementioned solvent are added to the system at approximately the same rate as distillate is formed.

In another embodiment of the present invention, the reactants (i.e., the lower-alkyl ester of α-hydroxy carboxylic acid 70 and the alcohol 75) are combined in a vessel of appropriate size and design such that controlled heating and stirring is allowed, and from which the reaction mixture can be easily removed. The ester 70 and catalyst (preferably an enzyme) 90 are combined such that the quantity of ester is an amount that represents a two-to-tenfold or higher molar excess based on the quantity of alcohol 75 which is to be used. The catalyst 90 is present in an amount that is between about 0.10 to about 2.0 times the weight of the alcohol 75 which is to be used. Optionally, a solvent is added in a volume of 100-1000 ml/mole of alcohol to be used. Suitable solvents include, but are not limited to, acetone, acetonitrile, dioxane, heptane, hexanes, and tetrahydrofuran. An absorbing agent (e.g., molecular sieves, silica gel, etc.) may be added in an amount that is between two to five times the weight of the alcohol 75. The reactants are stirred at an appropriate speed with controlled heating from about 30-90° C. while reducing the pressure in the system through the application of a weak vacuum. The alcohol 75 is added slowly by means of an addition funnel or syringe pump. The alcohol 85 generated during the reaction is removed by distillation or co-distillation with the aforementioned solvent. As needed, additional amounts of the aforementioned solvent are added to the system at approximately the same rate as distillate is formed.

In another embodiment of the present invention, the reactants (i.e., the lower-alkyl ester of α-hydroxy carboxylic acid 70 and the alcohol 75) are combined in a vessel of appropriate size and design such that controlled heating and stirring is allowed. The ester 70 and alcohol 75 are combined such that the quantity of ester used is an amount that represents a two-to-tenfold or higher molar excess based on the quantity of the alcohol. The mixture is maintained at about 50-90° C. while stirring, and liquid from the vessel is pumped continuously through a column packed with immobilized catalyst (preferably an enzyme) 90 and maintained at about 50-70° C., then returned to the vessel. Alcohol 85 produced during the reaction in the packed-bed column reactor is removed from the vessel through evaporation or distillation by reducing the pressure in the vessel by the application of a weak vacuum.

In another embodiment of the present invention, a mixture of alcohol 75 (X moles), ester 70 (1-5X moles), catalyst (preferably an enzyme) 90 (0.6×186.34X), and molecular sieves (5×186.34X) is placed in a stainless steel container of appropriate size such that the bed depth of the mixture does not exceed approximately 1.5″, with a preferred depth of approximately 1″. The container is placed in a convection oven at 60±5° C. for a time sufficient to consume greater than 95% of the alcohol 75. The time required is dependent upon the reaction size, and may be determined by monitoring the reaction periodically by High Performance Liquid Chromatography (HPLC) or Gas Chromatography (GC).

In another embodiment of the present invention, a mixture of alcohol 75 (1-dodecanol, X moles), ester 70 (ethyl lactate, 5X moles), catalyst 90 (the enzyme Novozym® 435, 0.6×186.34X), and molecular sieves (5×186.34X) is placed in a stainless steel container of appropriate size such that the bed depth of the mixture does not exceed approximately 1.5″, with a preferred depth of approximately 1″. The container is placed in a convection oven at 60° C. for a time sufficient to consume greater than 98% of the 1-dodecanol. The time required is dependent upon the reaction size, and may be determined by monitoring the reaction periodically by High Performance Liquid Chromatography—Refractive Index (HPLC-RI). Once the reaction has progressed satisfactorily, the container is removed from the oven and, after cooling to ambient temperature, the contents of the container are transferred to a larger container for the purpose of adding solvent. Adequate solvent is added to the container so that most of the product is solubilized. Solvents may be chosen from the group of lower molecular weight hydrocarbons, which may include, but are not limited to, pentane, petroleum ether, hexanes, heptane and isooctane. The solution of product in the hydrocarbon solvent is isolated by vacuum filtration, and transferred to a separatory funnel of adequate size to allow washing with an equal volume of water. The water wash is repeated two more times, and then the organic phase is washed with brine. The final organic solution is dried over magnesium sulfate to remove residual water. At this point, activated carbon may be added so as to render the solution nearly colorless. The dried, carbon-treated solution is then isolated by vacuum filtration employing a filtration aid, preferably Celite®. The solution is then concentrated by rotary evaporation until most of the solvent has been removed. Residual solvent can thereafter be removed by storing the product under vacuum, preferably 29″ Hg, preferably at a temperature not to exceed 30° C.

In another embodiment of the present invention, a multi-kilogram batch of lauryl lactate is produced in the absence of molecular sieves. The process involves a mixture of alcohol 75 (1-dodecanol, 4000 mL), ester 70 (ethyl lactate, 4000 mL, 2 molar equivalents), and catalyst 90 (the enzyme Novozym® 435, 1000 g). The mixture is placed in a 50 L glass pilot plant reactor and is stirred at 60±5° C. for 28-32 hours. Note: the specific time required may be determined by monitoring the reaction periodically by HPLC-RI or Gas Chromatography—Flame Ionization Detector (GC-FID). Once the reaction has progressed satisfactorily, the reaction mixture is filtered through a semi-permeable nylon bag to isolate the catalyst 90, and the filtrate is returned to the reactor where it is diluted with petroleum ether (4 L). The mixture is washed with brine (2 L) by adding the brine to the reactor, stirring for 1-2 minutes, allowing phase separation to occur, and then drawing off the lower aqueous phase to waste. The brine wash is repeated, and then the mixture is washed with water (4×2 L) in similar fashion. After the final water wash, the mixture is washed again with brine (2 L). Then MgSO₄ (100 g) and activated carbon (50 g) are added to the washed organic mixture, and the mixture is stirred for 1 hour at room temperature. The mixture is filtered through a glass fiber filter and the filtrate is concentrated by rotary evaporation until most of the solvent has been removed. Residual solvent can thereafter be removed by storing the product under vacuum, preferably 29″ Hg, preferably at a temperature not to exceed 30° C.

The following non-limiting examples provide some preferred methods for preparing fatty alcohol esters of α-hydroxy carboxylic acids (e.g., lauryl lactate, lauryl mandelate, 3,7-dimethyl-1-octyl lactate, cetyl lactate. The specific examples that follow are representative of the potential of the present invention and are not intended to be construed as limiting the invention in scope.

### EXAMPLE 1

A mixture of enzyme (275.07 g; lipase B from Candida Antarctica immobilized on macroporous acrylic resin beads; Novozym® 435), molecular sieves (2301 g), 1-dodecanol (551 mL), and ethyl lactate (1400 mL), in a 12“x20” stainless steel tray (bed depth 1.25″), was maintained at 60° C. in a convection oven for 32 hours. As a result of the foregoing, 538 g of lauryl lactate (83% yield) was obtained as a pale yellow liquid. The purity of the product was >95% as assessed by HPLC-RI.

### EXAMPLE 2

A mixture of Novozym® 435 (2.21 g), molecular sieves (18.60 g), 1-dodecanol (3.76 g), and ethyl mandelate (3.61 g), in a 150 mL beaker, was maintained at 60° C. in a convection oven for 28 hours. As a result of the foregoing, 4.0 g of lauryl mandelate (56% yield) was obtained as a clear viscous liquid. The purity of the product was >95% as assessed by High Performance Liquid Chromotography—Diode Array Detector (HPLC-DAD).

### EXAMPLE 3

A mixture of Novozym® 435 (2.81 g), molecular sieves (23.17 g), 3,7-dimethyl-1-octanol (4.64 g), and ethyl lactate (14.78 g), in a 250 mL beaker, was maintained at 60° C. in a convection oven for 28 hours. As a result of the foregoing, 4.2 g of 3,7-dimethyl-1-octyl lactate (62% yield) was obtained as an amber-colored liquid. The purity of the product was >96% as assessed by HPLC-RI.

### EXAMPLE 4

A mixture of Novozym® 435 (2.43 g), molecular sieves (20.17 g), 1-octadecanol (4.04 g), and ethyl lactate (8.86 g), in a 150 mL beaker, was maintained at 60° C. in a convection oven for 28 hours. As a result of the foregoing, 2.0 g of cetyl lactate (40% yield) was obtained as a waxy white solid. The purity of the product was >98% as assessed by HPLC-RI.

### EXAMPLE 5

A mixture of Novozym® 435 (1 kg), 1-dodecanol (2000 mL), and ethyl lactate (2000 mL), in a 50 L reactor, was stirred (˜200 rpm) at 60±5° C. for 28 hours. As a result of the foregoing, 2000 g of lauryl lactate (80% yield) was obtained as a pale yellow liquid. The purity of the product was >97% as assessed by GC.

### EXAMPLE 6

A mixture of Novozym® 435 (8.02 g), 1-dodecanol (13.01 g), ethyl lactate (8.70 mL) and acetonitrile (8.70 mL) was placed in a 100 mL three-neck, round bottom flask containing a stir bar. The flask was equipped with an addition funnel charged with acetonitrile, a stopper, and a short path distillation head, which was connected to a vacuum pump. The flask was placed in an oil bath, and the mixture was magnetically stirred at 80±5° C. Additional acetonitrile was added as needed based upon the amount of distillate collected. After 42 hours, analysis of the reaction mixture by GC-FID indicated that the purity of the lauryl lactate was >96%.

### EXAMPLE 7

A mixture of Novozym® 435 (8.01 g), 1-dodecanol (18.63 g), and hexanes (50 mL) was placed in a 250 mL three-neck, round bottom flask containing a stir bar. The flask was equipped with an addition funnel charged with ethyl lactate (25 mL), a stopper, and a short path distillation head, which was connected to a vacuum pump. The flask was placed in an oil bath, and the mixture was magnetically stirred at 80±5° C. As distillate began to collect, a slow dropwise addition of ethyl lactate was begun. Additional hexanes were added as needed. After 42 hours, analysis of the reaction mixture by GC-FID indicated that the purity of the lauryl lactate was >95%.

### EXAMPLE 8

A mixture of Novozym® 435 (5.99 g), 1-dodecanol (14.01 g), and hexanes (40 mL) was placed in a 250 mL three-neck, round bottom flask containing a stir bar. The flask was equipped with an addition funnel charged with hexanes, a rubber septum, and a short path distillation head, which was connected to a vacuum pump via an acetone-dry ice condenser. Ethyl lactate (15 mL) was drawn into a 20 mL syringe, which was secured in a syringe pump. The pump was programmed to deliver 10 mL of ethyl lactate at a rate of 1.0 mL/h. The syringe needle was inserted through the rubber septum, and the flask was placed in an oil bath. The mixture was magnetically stirred at 80±5° C. As distillate began to collect, the addition of ethyl lactate was begun. Hexanes were added dropwise at a rate approximating the rate at which distillate was collected. After 12 hours, all devices were turned off and the reaction mixture stood overnight at room temperature. Analysis of the reaction mixture by GC-FID indicated that the purity of the lauryl lactate was >95%.

### EXAMPLE 9

Novozym® 435 (250-300 g), molecular sieves (2250-2350 g), 1-dodecanol (540-560 mL), and ethyl lactate (1350-1450 mL) were mixed together in a 12“x20” stainless steel tray (bed depth ˜1.25″). A total of four trays were prepared in parallel, and maintained at 60° C. in a convection oven for 32 hours. After cooling to ambient temperature, the contents of each tray were diluted with petroleum ether, and the enzyme/sieves mixture was removed by vacuum filtration. The combined filtrates were transferred to a 50 L reactor and processed accordingly. As a result of the foregoing, 2024 g of lauryl lactate (78% yield) was obtained as a pale yellow liquid. The purity of the product was >98% as assessed by HPLC-RI.

### EXAMPLE 10

A mixture of Novozym® 435 (500 g), 1-dodecanol (2000 mL), and ethyl lactate (2000 mL), in a 5 L reactor, was stirred (˜200 rpm) at 60±5° C. for 28 hours. As a result of the foregoing, 2000 g of lauryl lactate (>80% yield) was obtained as a pale yellow liquid. The purity of the product was >95% as assessed by GC-FID.

### EXAMPLE 11

A mixture of Novozym® 435 (1 kg), 1-dodecanol (4000 mL), and ethyl lactate (4000 mL), in a 50 L reactor, was stirred (˜200 rpm) at 60±5° C. for 28 hours. As a result of the foregoing, 4000 g of lauryl lactate (80% yield) was obtained as a pale yellow liquid. The purity of the product was >97% as assessed by GC.

### CHRYSTAPHYL 98™

Thus, it will be seen that we have developed a convenient, efficient, and scalable process for the synthesis of high-purity alkyl lactates via transesterification.

In one preferred form of the invention, the high-purity alkyl lactate comprises high-purity lauryl lactate (>95%, verified by GC-FID) synthesized via transesterification of ethyl lactate with 1-dodecanol catalyzed by an immobilized enzyme lipase from Candida Antarctica (commercially available under the trade name Novozym® 435). This high-purity lauryl lactate product is currently commercially available from Chemic Laboratories, Inc. of Canton, Mass. under the trade name CHRYSTAPHYL 98™

### Topical Gel For The Transdermal Delivery Of Steroids

The preceding disclosure teaches a convenient, efficient, and scalable process for the synthesis of high-purity fatty alcohol esters via transesterification of a lower-alkyl ester of an α-hydroxy carboxylic acid. These high-purity fatty alcohol esters include alkyl lactates (and particularly lauryl lactate, cetyl lactate and myristyl lactate) and have particularly advantageous application as permeation enhancers for the transdermal delivery of drugs.

Thus, in another aspect of the present invention, there is provided a novel topical gel which comprises a high-purity fatty alcohol ester and a steroid, with the high-purity fatty alcohol ester acting as a permeation enhancer to facilitate the transdermal delivery of steroids.

And in another aspect of the present invention, there is provided a novel topical gel which comprises a high-purity alkyl lactate and a steroid, with the high-purity alkyl lactate acting as a permeation enhancer to facilitate the transdermal delivery of steroids.

In one preferred form of the invention, there is provided a novel topical gel which comprises the aforementioned high-purity lauryl lactate (>95%) and a steroid, with the high-purity lauryl lactate acting as a permeation enhancer to facilitate the transdermal delivery of the steroid.

More particularly, in one preferred form of the invention, the topical gel comprises the aforementioned high-purity lauryl lactate and testosterone, with the high-purity lauryl lactate acting as a permeation enhancer for the testosterone. The following examples illustrate how such a gel may be formulated and prepared.

### EXAMPLE 12

Testosterone was weighed into a 250 mL tall form beaker. Ethanol (190 proof) was added to the beaker followed by water, and then CHRYSTAPHYL 98™ was added. Brief sonication gave a clear solution. Carbomer® (Carbopol® 980) was sprinkled into the solution as it was stirred via overhead stirring (600-700 rpm). After all of the carbomer had been homogeneously dispersed, stir speed was increased to ˜1200 rpm. After several hours, a homogeneous, translucent, viscous solution had formed. Sodium hydroxide solution (1.0 N) was introduced below the surface of the solution, and stirring was continued for 4-5 hours until gelling was complete.

Process constituents were provided in the following amounts:

| Constituent | % w/w |
|---|---|
| Ethanol | 67.37 |
| Water | 24.93 |
| Chrystaphyl-98 ™ | 4.75 |
| Testosterone | 0.99 |
| Carbopol ® 980 | 1.95 |
| NaOH | ˜0.01 |
| | 100.00 |

### EXAMPLE 13

Testosterone was weighed into a 250 mL tall form beaker. Ethanol (190 proof) was added to the beaker followed by water, and then CHRYSTAPHYL 98™ was added. Brief sonication gave a clear solution. Carbomer® (Carbopol® 980) was sprinkled into the solution as it was stirred via overhead stirring (600-700 rpm). After all of the carbomer had been homogeneously dispersed, stir speed was increased to ˜1200 rpm. After several hours, a homogeneous, translucent, viscous solution had formed. Sodium hydroxide solution (1.0 N) was introduced below the surface of the solution, and stirring was continued for 4-5 hours until gelling was complete.

Process constituents were provided in the following amounts:

| Constituent | % w/w |
|---|---|
| Ethanol | 66.01 |
| Water | 21.18 |
| Chrystaphyl-98 ™ | 9.88 |
| Testosterone | 0.99 |
| Carbopol ® 980 | 1.93 |
| NaOH | ˜0.01 |
| | 100.00 |

### EXAMPLE 14

Testosterone was weighed into a 250 mL tall form beaker. Ethanol (190 proof) was added to the beaker followed by water, and then CHRYSTAPHYL 98™ was added. Brief sonication gave a clear solution. Carbomer® (Carbopol® 980) was sprinkled into the solution as it was stirred via overhead stirring (600-700 rpm). After all of the carbomer had been homogeneously dispersed, stir speed was increased to ˜1200 rpm. After several hours, a homogeneous, translucent, viscous solution had formed. Sodium hydroxide solution (1.0 N) was introduced below the surface of the solution, and stirring was continued for 4-5 hours until gelling was complete.

Process constituents were provided in the following amounts:

| Constituent | % w/w |
|---|---|
| Ethanol | 68.62 |
| Water | 18.35 |
| Chrystaphyl-98 ™ | 5.03 |
| Isopropyl myristate | 5.00 |
| Testosterone | 1.00 |
| Carbopol ® 980 | 1.99 |
| NaOH | ˜0.01 |
| | 100.00 |

### Additional Gel Formulations

It should be appreciated that various formulations and processes can be used to produce the novel topical gel of the present invention. Among other things, high-purity permeation enhancers other than lauryl lactate may be used, e.g., cetyl lactate, myristyl lactate, other high-purity alkyl lactates and other high-purity fatty alcohol esters, and mixtures or blends of two or more of the foregoing may be used. Similarly, steroids other than testosterone may be used in conjunction with the high-purity permeation enhancers to form the novel topical gel. More specifically, any steroid which will dissolve in a gel formulation made in accordance with the present invention can be used in the novel topical gel.

### EXAMPLE 15

### (0.5% Testosterone Gel)

Testosterone was weighed into a 200 mL tall form beaker, followed by ethanol (190 or 200 proof). Water was added to the resultant clear solution, followed by lauryl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 200 | | 25.568 | 67.35 |
| 2 | water | | 9.284 | 24.94 |
| 3 | lauryl lactate | | 1.802 | 4.75 |
| 4 | testosterone | | 0.374 | 0.99 |
| 5 | Carbopol 980 | | 0.741 | 1.95 |
| 6 | NaOH, 1 N | 184 | 0.191 | |
| 7 | NaOH | | 0.008 | 0.02 |
| 8 | water | | 0.184 | |
| | | | 37.960 | 100.00 |

### EXAMPLE 16

### (0.5% Testosterone Gel)

Testosterone was weighed into a 200 mL tall form beaker, followed by ethanol (190 or 200 proof). Water was added to the resultant clear solution, followed by lauryl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 28.20 | |
| | water | | 1.73 | 25.20 |
| | EtOH | | 26.47 | 67.21 |
| 2 | water | | 7.99 | |
| 3 | lauryl lactate | | 1.99 | 5.05 |
| 4 | testosterone | | 0.201 | 0.51 |
| 5 | Carbopol 980 | | 0.79 | 2.01 |
| 6 | NaOH, 1 N | 200 | 0.21 | |
| | NaOH | | 0.01 | 0.02 |
| | water | | 0.20 | |
| | | | 39.38 | 100.00 |

### EXAMPLE 17

### (0.5% Testosterone Gel)

Testosterone was weighed into a 200 mL tall form beaker, followed by ethanol (190 or 200 proof). Water was added to the resultant clear solution, followed by myristyl lactate. Carbomer® (Carbopol® 980) was sprinkled into the beaker while the mixture was mechanically stirred at high speed. After all of the carbomer was homogeneously dispersed, NaOH was added and stirring was continued until gel formation was

The process constituents were provided in the following amounts:

| | Ingredient | mL | wt, g | % w/w |
|---|---|---|---|---|
| 1 | EtOH, 190 | | 24.83 | |
| | water | | 1.53 | 25.23 |
| | EtOH | | 23.30 | 67.17 |
| 2 | water | | 7.03 | |
| 3 | myristyl lactate | | 1.75 | 5.04 |
| 4 | testosterone | | 0.176 | 0.51 |
| 5 | Carbopol 980 | | 0.70 | 2.02 |
| 6 | NaOH, 1 N | 200 | 0.21 | |
| | NaOH | | 0.01 | 0.02 |
| | water | | 0.20 | |
| | | | 34.69 | 100.00 |

### EXAMPLE 18

### (0.5% Testosterone Gel)

Testosterone was weighed into a 200 mL tall form beaker, followed by ethanol (190 or 200 proof). Water was added to the resultant clear solution, followed by cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 28.21 | |
| | water | | 1.73 | 25.20 |
| | EtOH | | 26.48 | 67.24 |
| 2 | water | | 7.99 | |
| 3 | cetyl lactate | | 1.99 | 5.05 |
| 4 | testosterone | | 0.202 | 0.51 |
| 5 | Carbopol 980 | | 0.78 | 1.98 |
| 6 | NaOH, 1 N | 200 | 0.21 | |
| | NaOH | | 0.01 | 0.02 |
| | water | | 0.20 | |
| | | | 39.38 | 100.00 |

### EXAMPLE 19

### (0.5% Testosterone Gel)

Testosterone was weighed into a 200 mL tall form beaker, followed by ethanol (190 or 200 proof). Water was added to the resultant clear solution, followed by lauryl lactate and cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 28.20 | |
| | water | | 1.73 | 25.20 |
| | EtOH | | 26.47 | 67.23 |
| 2 | water | | 7.99 | |
| 3 | lauryl lactate | | 1.00 | 2.54 |
| 4 | cetyl lactate | | 0.98 | 2.49 |
| 5 | testosterone | | 0.200 | 0.51 |
| 6 | Carbopol 980 | | 0.79 | 2.01 |
| 7 | NaOH, 1 N | 200 | 0.21 | |
| | NaOH | | 0.01 | 0.02 |
| | water | | 0.20 | |
| | | | 39.37 | 100.00 |

### EXAMPLE 20

### (0.5% Testosterone Gel)

Testosterone was weighed into a 200 mL tall form beaker followed by ethanol (190 or 200 proof). Water was added to the resultant clear solution, followed by myristyl lactate and cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 28.20 | |
| | water | | 1.73 | 25.18 |
| | EtOH | | 26.47 | 67.18 |
| 2 | water | | 7.99 | |
| 3 | myristyl lactate | | 1.00 | 2.54 |
| 4 | cetyl lactate | | 1.00 | 2.54 |
| 5 | testosterone | | 0.199 | 0.51 |
| 6 | Carbopol 980 | | 0.80 | 2.03 |
| 7 | NaOH, 1 N | 200 | 0.21 | |
| | NaOH | | 0.01 | 0.02 |
| | water | | 0.20 | |
| | | | 39.40 | 100.00 |

### EXAMPLE 21

### (0.5% Testosterone Gel)

Testosterone was weighed into a 200 mL tall form beaker, followed by ethanol (190 or 200 proof). Water was added to the resultant clear solution, followed by lauryl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 200 | | 25.568 | 67.35 |
| 2 | water | | 9.284 | 24.94 |
| 3 | lauryl lactate | | 1.802 | 4.75 |
| 4 | testosterone | | 0.374 | 0.99 |
| 5 | Carbopol 980 | | 0.741 | 1.95 |
| 6 | NaOH, 1 N | 184 | 0.191 | |
| 7 | NaOH | | 0.008 | 0.02 |
| 8 | water | | 0.184 | |
| | | | 37.960 | 100.00 |

### EXAMPLE 22

### (0.5% Testosterone Gel)

Testosterone was weighed into a 200 mL tall form beaker followed by ethanol (190 or 200 proof). Water was added to the resultant clear solution, followed by lauryl lactate and cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 28.20 | |
| | water | | 1.73 | 25.20 |
| | EtOH | | 26.47 | 67.23 |
| 2 | water | | 7.99 | |
| 3 | lauryl lactate | | 1.00 | 2.54 |
| 4 | cetyl lactate | | 0.98 | 2.49 |
| 5 | testosterone | | 0.200 | 0.51 |
| 6 | Carbopol 980 | | 0.79 | 2.01 |
| 7 | NaOH, 1 N | 200 | 0.21 | |
| | NaOH | | 0.01 | 0.02 |
| | water | | 0.20 | |
| | | | 39.37 | 100.00 |

### EXAMPLE 23

### (0.5% Testosterone Gel)

Testosterone was weighed into a 200 mL tall form beaker, followed by ethanol (190 or 200 proof). Water was added to the resultant clear solution, followed by lauryl lactate and cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 14.10 | |
| | water | | 0.87 | 25.20 |
| | EtOH | | 13.23 | 67.08 |
| 2 | water | | 4.01 | |
| 3 | lauryl lactate | | 0.42 | 2.13 |
| 4 | cetyl lactate | | 0.60 | 3.04 |
| 5 | testosterone | | 0.100 | 0.51 |
| 6 | Carbopol 980 | | 0.40 | 2.03 |
| 7 | NaOH, 1 N | 100 | 0.10 | |
| | NaOH | | 0.00 | 0.02 |
| | water | | 0.10 | |
| | | | 19.73 | 100.00 |

### EXAMPLE 24

### (0.5% Testosterone Gel)

Testosterone was weighed into a 200 mL tall form beaker, followed by ethanol (190 or 200 proof). Water was added to the resultant clear solution, followed by lauryl lactate and cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 14.10 | |
| | water | | 0.87 | 25.20 |
| | EtOH | | 13.23 | 67.08 |
| 2 | water | | 4.01 | |
| 3 | lauryl lactate | | 0.30 | 1.52 |
| 4 | cetyl lactate | | 0.72 | 3.65 |
| 5 | testosterone | | 0.099 | 0.50 |
| 6 | Carbopol 980 | | 0.40 | 2.03 |
| 7 | NaOH, 1 N | 100 | 0.10 | |
| | NaOH | | 0.00 | 0.02 |
| | water | | 0.10 | |
| | | | 19.73 | 100.00 |

### EXAMPLE 25

### (0.5% Testosterone Gel)

Testosterone was weighed into a 200 mL tall form beaker, followed by ethanol (190 or 200 proof). Water was added to the resultant clear solution, followed by lauryl lactate and myristyl lactate. Carbomer® Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 12.05 | |
| | water | | 0.74 | 25.22 |
| | EtOH | | 11.31 | 67.18 |
| 2 | water | | 3.41 | |
| 3 | lauryl lactate | | 0.17 | 1.01 |
| 4 | myristyl lactate | | 0.68 | 4.04 |
| 5 | testosterone | | 0.086 | 0.51 |
| 6 | Carbopol 980 | | 0.34 | 2.02 |
| 7 | NaOH, 1 N | 85 | 0.10 | |
| | NaOH | | 0.00 | 0.02 |
| | water | | 0.10 | |
| | | | 16.84 | 100.00 |

### EXAMPLE 26

### (0.5% Testosterone Gel)

Testosterone was weighed into a 200 mL tall form beaker followed by ethanol (190 or 200 proof). Water was added to the resultant clear solution, followed by lauryl lactate and cetyl lactate. Carbomer® (Carbopol® 980) was sprinkled into the beaker while the mixture was mechanically stirred at high speed. After all of the carbomer was homogeneously dispersed, NaOH was added and stirring was continued until gel formation was complete.

The process constituents were provided in the following amounts:

| | Ingredient | mL | wt, g | % w/w |
|---|---|---|---|---|
| 1 | EtOH, 190 | | 10.11 | |
| | water | | 0.62 | 20.30 |
| | EtOH | | 9.49 | 66.96 |
| 2 | water | | 2.16 | |
| 3 | lauryl lactate | | 0.41 | 2.89 |
| 4 | cetyl lactate | | 1.03 | 7.27 |
| 5 | testosterone | | 0.072 | 0.51 |
| 6 | Carbopol 980 | | 0.29 | 2.05 |
| 7 | NaOH, 1 N | 72 | 0.10 | |
| | NaOH | | 0.00 | 0.03 |
| | water | | 0.10 | |
| | | | 14.17 | 100.00 |

Additionally, other gel formulations can be envisioned in which the relative percentages of ingredients are varied. Likewise, other gel formulations can be envisioned in which the basifying agent is changed, e.g., a different concentration of sodium hydroxide is employed, or an entirely different base is used (e.g., potassium hydroxide, or an organic amine), etc.

### Efficacy

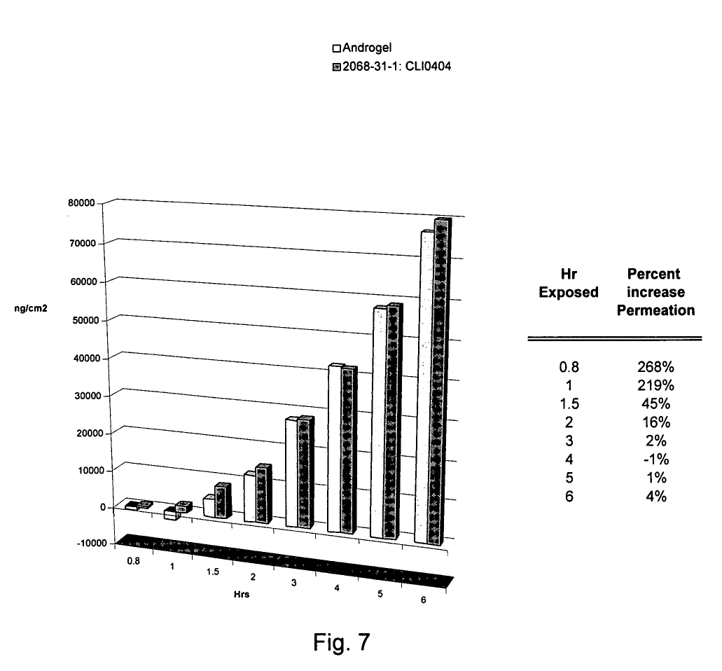

The high-purity fatty alcohol esters formed in accordance with the present invention provide permeation enhancers of superior performance. FIG. 7 illustrates how a testosterone gel employing CHRYSTAPHYL 98™ as the permeation enhancer provided improved transdermal delivery of the testosterone.

Permeation study protocol parameters employed were based upon those methods described by Skelly (1987), Anon. (1993) and Scott & Clowes. More particularly, in order to evaluate new chemical entities as putative permeation enhancers, in vitro diffusion studies, e.g., Franz cell studies, are performed. Ideally these studies are conducted using human skin.

A study was conducted to evaluate Chemic's CHRYSTAPHYL 98™ brand of high-purity lauryl lactate as a permeation enhancer, and compare its efficacy with that of isopropyl myristate for the transdermal delivery of testosterone. The studies utilized a modified Franz cell apparatus and a stratum corneum model system, i.e., the EpiDerm™ 606X Skin Model (MatTek® Inc.). This model exhibits in vivo-like morphological and growth characteristics, which are uniform and highly reproducible. EpiDerm™ consists of organized basal, spinous, granular, and cornified layers analogous to those found in vivo, and is mitotically and metabolically active. Ultrastructural analysis has revealed the presence of keratohyalin granules, tonofilament bundles, desmosomes, and a multi-layered stratum corneum containing intercellular lamellar lipid layers arranged in patterns characteristic of in vivo epidermis. The morphological characterization of the MatTek® product and its comparability to in vivo stratum corneum supports the use of the product as an in vitro model system. The use of testosterone as the pharmacologically active agent allowed the direct utilization of literature values, and provided the basis for the direct comparison of Chemic's testosterone gel formulation with Androgel® (testosterone gel) 1%.

### Modifications

It is to be understood that the present invention is by no means limited to the particular constructions herein disclosed and/or shown in the drawings, but also comprises any modifications or equivalents within the scope of the invention.

What is claimed is:

1. A gel comprising:
a steroid; and
a permeation enhancer to facilitate the transdermal delivery of the steroid, wherein the permeation enhancer comprises a high-purity fatty alcohol ester.

2. A gel according to claim 1 wherein the high-purity fatty alcohol ester has a purity >95%.

3. A gel according to claim 1 wherein the high-purity fatty alcohol ester comprises a high-purity alkyl lactate.

4. A gel according to claim 3 wherein the high-purity alkyl lactate comprises lauryl lactate.

5. A gel according to claim 3 wherein the high-purity alkyl lactate comprises cetyl lactate.

6. A gel according to claim 3 wherein the high-purity alkyl lactate comprises myristyl lactate.

7. A gel according to claim 1 wherein the high-purity fatty alcohol ester comprises lauryl mandelate.

8. A gel according to claim 1 wherein the high-purity fatty alcohol ester comprises 3,7-dimethyl-1-octyl lactate.

9. A gel according to claim 1 wherein the fatty alcohol ester is derived from an α-hydroxy carboxylic acid.

10. A gel according to claim 9 wherein the fatty alcohol ester is formed by converting a lower-alkyl ester of α-hydroxy carboxylic acid into a fatty alcohol ester of α-hydroxy carboxylic acid via transesterification, and further wherein the transesterification process is an equilibrium reaction that is shifted in the desired direction to produce the desired product.

11. A gel according to claim 9 wherein the fatty alcohol ester is formed by (i) providing a lower-alkyl ester of an α-hydroxy carboxylic acid, an alcohol, and a catalyst; and (ii) converting the lower-alkyl ester of the α-hydroxy carboxylic acid into a fatty alcohol ester through transesterification, and wherein the transesterification process is an equilibrium reaction that is shifted in the desired direction to produce the desired product.

12. A gel according to claim 11 wherein the transesterification process is catalyzed chemically.

13. A gel according to claim 12 wherein the transesterification process is catalyzed with an acid.

14. A gel according to claim 12 wherein the transesterification process is catalyzed with a base.

15. A gel according to claim 11 wherein the transesterification process is catalyzed with an enzyme.

16. A gel according to claim 15 wherein the enzyme is a lipase.

17. A gel according to claim 16 wherein the lipase is obtained from a microorganism selected from the group consisting of: Aspergillus species, Rhizopus species, Penicillum species, Candida species, Pseudomonas species, Mucor species, and Humicola species.

18. A gel according to claim 1 wherein the permeation enhancer comprises a blend of two or more high-purity fatty alcohol esters.

19. A gel according to claim 1 wherein the steroid is testosterone.

20. A gel according to claim 1 wherein the steroid is any steroid which is soluble in the gel.

21. A topical transdermal drug delivery product comprising:
a drug; and
a permeation enhancer to facilitate the transdermal delivery of the drug, wherein the permeation enhancer comprises a high-purity fatty alcohol ester.

22. A topical transdermal drug delivery product according to claim 21 wherein the drug comprises a steroid.

23. A topical transdermal drug delivery product according to claim 22 wherein the steroid comprises testosterone.

24. A topical transdermal drug delivery product according to claim 21 wherein the high-purity fatty alcohol ester has a purity >95%.

25. A topical transdermal drug delivery product according to claim 21 wherein the high-purity fatty alcohol ester comprises a high-purity alkyl lactate.

26. A topical transdermal drug delivery product according to claim 25 wherein the high-purity alkyl lactate comprises lauryl lactate.

27. A topical transdermal drug delivery product according to claim 25 wherein the high-purity alkyl lactate comprises cetyl lactate.

28. A topical transdermal drug delivery product according to claim 25 wherein the high-purity alkyl lactate comprises myristyl lactate.

29. A topical transdermal drug delivery product according to claim 21 wherein the high-purity fatty alcohol ester comprises lauryl mandelate.

30. A topical transdermal drug delivery product according to claim 21 wherein the high-purity fatty alcohol ester comprises 3,7-dimethyl-1-octyl lactate.

31. A topical transdermal drug delivery product according to claim 21 wherein the fatty alcohol ester is derived from an α-hydroxy carboxylic acid.

32. A topical transdermal drug delivery product according to claim 31 wherein the fatty alcohol ester is formed by converting a lower-alkyl ester of α-hydroxy carboxylic acid into a fatty alcohol ester of α-hydroxy carboxylic acid via transesterification, and further wherein the transesterification process is an equilibrium reaction that is shifted in the desired direction to produce the desired product.

33. A topical transdermal drug delivery product according to claim 31 wherein the fatty alcohol ester is formed by (i) providing a lower-alkyl ester of an α-hydroxy carboxylic acid, an alcohol, and a catalyst; and (ii) converting the lower-alkyl ester of the α-hydroxy carboxylic acid into a fatty alcohol ester through transesterification, and wherein the transesterification process is an equilibrium reaction that is shifted in the desired direction to produce the desired product.

34. A method for manufacturing a transdermal steroid delivery product, comprising:
(i) providing a steroid and providing a permeation enhancer to facilitate the transdermal delivery of the steroid, wherein the permeation enhancer comprises a high-purity fatty alcohol ester; and
(ii) combining the steroid and the fatty alcohol ester in a gel.

35. A method for manufacturing a transdermal drug delivery product, comprising:
(i) providing a drug and providing a permeation enhancer to facilitate the transdermal delivery of the drug, wherein the permeation enhancer comprises a high-purity fatty alcohol ester; and
(ii) combining the drug and the fatty alcohol ester in a mass selected from the group consisting of a gel, a cream, an ointment, a liquid, a colloid, a semi-solid, and a solid.

36. A method for delivering a steroid to living tissue, comprising:
(i) providing a gel comprising (a) a steroid, and (b) a permeation enhancer to facilitate the transdermal delivery of the steroid, wherein the permeation enhancer comprises a high-purity fatty alcohol ester; and
(ii) applying the gel to the living tissue.

37. A method for delivering a drug to living tissue, comprising:
(i) providing a mass comprising (a) a drug, and (b) a permeation enhancer to facilitate the transdermal delivery of the drug, wherein the permeation enhancer comprises a high-purity fatty alcohol ester, and further wherein the mass is selected from the group consisting of a gel, a cream, an ointment, a liquid, a colloid, a semi-solid, and a solid; and
(ii) applying the mass to the living tissue.
